(19) United States
(12) Patent Application Publication
(10) Pub. No.: US 2023/0190411 A1
(43) Pub. Date: Jun. 22, 2023

(54) TREATMENT PLANNING USING TOOTH MOVEMENT MODELING

(71) Applicant: Align Technology, Inc., San Jose, CA (US)

(72) Inventors: Yi-Lin CHIU, Foster City, CA (US); Yuxiang WANG, Newark, CA (US); Luyao CAI, San Jose, CA (US); Jun SATO, San Jose, CA (US); Iman SHOJAEI, Fremont, CA (US); Manlio Fabio VALDIVIESO CASIQUE, Santa Clara, CA (US); Minghao DAI, Toronto (CA); Xi CAI, San Jose, CA (US); Jeeyoung CHOI, Sunnyvale, CA (US); Eric YAU, Saratoga, CA (US)

(21) Appl. No.: 18/067,595

(22) Filed: Dec. 16, 2022

Related U.S. Application Data
(60) Provisional application No. 63/291,318, filed on Dec. 17, 2021.

(51) Int. Cl. A61C 7/00; A61C 13/34; G16H 10/60; G16H 20/40

(52) U.S. Cl. CPC A61C 7/002; A61C 13/34; G16H 10/60; G16H 20/40

(57) ABSTRACT

Methods and apparatuses (including software) for optimizing dental treatment plans, including for optimizing the treatment plans using dental aligners. These methods and apparatuses may optimize a treatment plan by estimating a difference between a target set of tooth position and a predicted set of tooth positions using the treatment plan using a prediction network trained to use multiple translational and rotational directions for individual teeth as well as reaction forces on the individual teeth based on adjacent teeth.

### CLAIM OF PRIORITY

This patent application claims priority to U.S. Provisional Patent Application No. 63/291,318, titled “TREATMENT PLANNING USING TOOTH MOVEMENT MODELING,” filed on Dec. 17, 2021 and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Orthodontic and dental treatments using a series of patient-removable appliances (e.g., “aligners”) are very useful for treating a variety of patients. Treatment planning is typically performed in conjunction with the dental professional (e.g., dentist, orthodontist, dental technician, etc.), by manipulating a model of the patient's teeth from an initial configuration to a final configuration and then dividing the treatment into a number of intermediate stages (steps). These steps may correspond to individual appliances that may be worn sequentially, with or without additional interventions (e.g., interproximal reductions, extractions, etc.). Once the treatment plan is finalized, the series of aligners may be manufactured corresponding to the treatment plan.

In practice, the actual tooth movements may differ from those proposed by a treatment plan, due to a variety of different causes. Thus, the patient's final tooth position during the course of a treatment plan may be different from what is initially proposed. It would be useful to provide methods and apparatuses (e.g., systems and devices, including software) accurately predicting tooth movement and for modifying a treatment plan accordingly.

### SUMMARY OF THE DISCLOSURE

Described herein are methods and apparatuses relevant to the design and execution of dental appliances, and in particular the design, formation and treatment of dental aligners for aligning a subject's teeth. The methods and apparatuses (e.g., devices and/or systems, including software and dental appliances) may be used for progress tracking, treatment optimization, tooth movement model, and customization of dental treatment planning.

One way to improve aligner therapy is to optimize a treatment plan by taking individual difference into account. The methods and apparatuses described herein may compute an optimal treatment plan by utilizing a customized tooth movement prediction model, along with a constrained optimization technique. These methods and apparatuses may build and/or use a machine learning agent (forming a trained prediction model) implementing a linear regression (LR) model, to predict tooth movement given a treatment plan, one or more patient characteristics (e.g., patient demographical info, including but not limited to age, gender, etc.) and in some examples the progress information (if available) from patients. Based on the prediction of the tooth movement, these methods and apparatuses may utilize a constrained optimization procedures to optimize a treatment plan having a higher aligner therapy efficacy.

These methods and apparatuses may provide enhanced safety for treatment planning, and may allow users (ex. doctors, technicians, dentists, orthodontists, dental professionals, etc.) to set weighting coefficients to boost or constrain the movements of the prescribed tooth set. The closed form solution may therefore be derived and may enables for the first time a low-cost, real-time processing for the treatment optimization based on predicted tooth movement.

The methods and apparatuses described herein may be particularly accurate and efficient in predicting tooth movement, because the trained prediction model (e.g., the Machine Learning agent or ML agent) may be consider not just movement of each individual tooth separately, but may consider multiple dimensions of movement of each individual tooth in combination with the effects of movement due to teeth that are adjacent to each individual tooth. Thus, these methods and apparatuses may use information from the tooth of prediction but also from multiple related teeth, which allows the model to account for complex tooth interactions (ex. reaction force) in the dentition. Surprisingly, in practice, including this feature increases the prediction accuracy from 20-30% to 70-80% or more. One substantial advantage of the methods and apparatuses described herein in comparison to other suggested approaches is the ability of these methods and apparatuses to account for individual variance, as well as the remarkable speed, accuracy and controllability in the optimization of treatment planning. The methods and apparatuses described herein also typically require less processing time and power as compared to other tooth movement prediction techniques.

For example, described herein are methods and apparatuses that may generally relate to the fabrication of dental appliances. Dental appliances may be fabricated based on predicted treatment plans optimized by comparing target tooth positions (that may be specified by a user, such as a dental practitioner) with predicted tooth positions that may be provided by a trained prediction model (e.g., machine learning model), as described herein. The trained prediction model may predict a set of final tooth positions of a patient's dental teeth based on a proposed treatment plan. In some cases, the trained prediction model may predict tooth positions (including final tooth positions) based, at least in part, on interactions between a target tooth and one or more adjacent teeth. The trained prediction model may be trained through analysis of historical patient data (training data) that may be generalized across patients or, in some examples, may be organized by patient characteristics (e.g., by demographic characteristics, clinical concerns, and/or cosmetic concerns). In some examples, the training data may be updated or modified based on a patient's own historical treatment data.

In some cases, a difference measurement, which may be referred to herein as a difference indicator, between a predicted set of tooth positions/orientations and desired final set of tooth positions/orientations can be made. If the difference indicator is less than a threshold, then the patient's dental appliances may be fabricated based on the treatment plan that generated the difference indicator. If the difference indicator is greater than or equal to the threshold, then the treatment plan may be adjusted (including iteratively adjusted), and a new predicted set of final tooth positions/orientations may be determined. Thus, the patient's treatment plan may be iterated until the difference indicator is less than the threshold. The difference indicator may be weighted based on general or specific (e.g., customized to a particular user/dental professional associated with the patient) weighting values in order to emphasize or de-emphasize particular dental features.

In some examples, the predicted set of final tooth positions may be displayed on a display, in some cases adjacent to and/or superimposed over the patient's set of initial tooth positions. The target tooth position may also be displayed adjacent to and/or superimposed over an image (the same or a different image) of the patient's set of initial tooth positions. Alternatively or additionally, the predicted set of final tooth positions (or any of the intermediate predicted sets of final tooth positions) may be displayed adjacent to and/or superimposed over the set of target tooth positions. Any of these display options may allow a dental professional and/or patient to easily visualize results of the proposed and/or modified dental treatment plan. Any of the methods and apparatuses described herein may be configured to display, e.g., on a user display, an image of the desired (target) final position of the patient's teeth and an image of a predicted final position of the patient's teeth. In some cases, the user display may be included in a mobile device, a laptop computer, or a tablet computer. In some other cases, determining the predicted tooth movement may be further based on the desired tooth movement of the patient.

As used herein the set of tooth positions may refer to the position and orientation of each of the patient's teeth in the set. The set (e.g., the set of initial patient tooth positions) may include all of the patient's teeth or a subset of the patient's teeth. The set of initial patient tooth positions may include the patient's teeth in the patient's upper and/or the patient's lower dental arch. The same teeth may be included in the set of initial patient tooth positions as the set of target tooth positions (though in some examples the set of target tooth positions may include fewer teeth, e.g., in the event of an extraction).

Also described herein are methods of generating dental appliance data to provide a dental treatment. The method may include determining a desired tooth movement of a patient to implement a treatment plan, wherein the desired tooth movement includes a desired final position of a patient's teeth, determining a predicted tooth movement of the patient based at least in part on a biomechanical interaction between teeth described in historic tooth movement data, wherein the predicted tooth movement includes a predicted final position of the patient's teeth, determining a difference between the desired final position of the patient's teeth and the predicted final position of the patient's teeth, and generating dental appliance data based on the desired tooth movement when the difference between the desired final position of the patient's teeth and the predicted final position of the patient's teeth is less than a threshold.

The biomechanical interaction between teeth may include an interaction between at least one target tooth and two teeth immediately adjacent to the one target tooth. In some examples, the biomechanical interaction between teeth may be modeled with a linear regression of tooth movement parameters of at least one target tooth and at least two teeth immediately adjacent to the one target tooth. The tooth movement parameters may include an amount of movement in six independent axes of motion. In some cases, the six independent axes of motion may include three linear axes and three rotational axes. The linear regression may include a Huber linear regression.

The method of generating dental appliance data may further include revising the desired tooth movement of the patient when the difference between the desired final position of the patient's teeth and the predicted final position of the patient's teeth is greater than the threshold, wherein generating the dental appliance data is based at least in part on the revised desired tooth movement. The desired tooth movement may be iteratively revised until the difference between the desired final position of the patient's teeth and the predicted final position of the patient's teeth is less than the threshold. In some examples, the revised desired tooth movement may include planned tooth positions having position parameters that position at least one tooth outside an associated dental arch that includes the desired final position of the patient's teeth. Further, the position parameters that position at least one tooth outside an associated dental arch may be within a range of safety limits for each position parameter. The safety limits may include a maximum difference between position parameters for a planned tooth position outside the associated dental arch and the desired final position of the patient's teeth.

In some examples, determining the difference between the desired final position of the patient's teeth and the predicted final position of the patient's teeth may include summing the difference between a position of each tooth in the desired final position of the patient's teeth and an associated tooth in the predicted final position of the patient's teeth. In some cases, determining the difference between the desired final position of the patient's teeth and the predicted final position of the patient's teeth may further include summing the difference in each of six independent axes of motion between each tooth in the desired final position of the patient's teeth and an associated tooth in the predicted final position of the patient's teeth. In some other cases, determining the difference between the desired final position of the patient's teeth and the predicted final position of the patient's teeth may further include preferentially weighting a position of particular teeth included in the summed difference between a position of each tooth in the desired final position of the patient's teeth and an associated tooth in the predicted final position of the patient's teeth. The weights of the positions of particular teeth may be determined based on a treatment target.

In some examples, determining the predicted tooth movement of the patient may be updated based on tooth movement data from a previous dental treatment of the patient. In some other examples, determining the predicted tooth movement of the patient may be based on patient age, patient gender, treatment type, or a combination thereof.

In some examples, the training set of prior tooth movement data used to train the trained prediction model may include desired tooth movement data and achieved tooth movement data for a plurality of patients. The trained prediction model may be continuously or repeatedly trained as the data set expands. In any of these examples, a library of different trained prediction models, indexed by patient characteristic(s), may be used. Thus the methods and apparatuses described herein may be customized by selecting a particular trained prediction model based on one or more patient characteristics.

Any of the methods and apparatuses described herein may include generating dental appliances based on the optimized treatment plans. Generating dental appliances may include generating instructions for manually, semi-automatically or automatically generating dental appliances, including a series of dental appliances. For example, these methods and apparatuses may cause the fabrication of dental appliances using, e.g., three-dimensional (3D) printing.

Also described herein are apparatuses (e.g., systems, devices, etc.) configured to perform any of the methods described herein. These apparatuses may include a non-transitory computer-readable storage medium. The non-transitory computer-readable storage medium may include instructions that, when executed by one or more processors of a device, cause the device to perform operations comprising the steps of these methods. For example, the non-transitory computer-readable storage medium may include instructions that, when executed by one or more processors of a device, cause the device to perform operations comprising the steps of determining a desired tooth movement of a patient to implement a treatment plan, wherein the desired tooth movement includes a desired final position of a patient's teeth, determining a predicted tooth movement of the patient based at least in part on a biomechanical interaction between teeth described in historic tooth movement data, wherein the predicted tooth movement includes a predicted final position of the patient's teeth, determining a difference between the desired final position of the patient's teeth and the predicted final position of the patient's teeth, and generating dental appliance data based on the desired tooth movement when the difference between the desired final position of the patient's teeth and the predicted final position of the patient's teeth is less than a threshold.

In some examples, the biomechanical interaction between teeth may include an interaction between at least one target tooth and two teeth immediately adjacent to the one target tooth. In some other examples, the biomechanical interaction between teeth may be modeled with a linear regression of tooth movement parameters of at least one target tooth and at least two teeth immediately adjacent to the one target tooth. The tooth movement parameters may include an amount of movement in six independent axes of motion. In some cases, the six independent axes of motion may include three linear axes and three rotational axes. In some other cases, the linear regression may be a Huber linear regression.

The execution of the instructions may cause the device to perform operations including revising the desired tooth movement of the patient when the difference between the desired final position of the patient's teeth and the predicted final position of the patient's teeth is greater than the threshold, wherein generating the dental appliance data is based at least in part on the revised desired tooth movement. In some examples, the desired tooth movement may be iteratively revised until the difference between the desired final position of the patient's teeth and the predicted final position of the patient's teeth is less than the threshold. In some other examples, the revised desired tooth movement may include planned tooth positions having position parameters that position at least one tooth outside an associated dental arch that includes the desired final position of the patient's teeth. The position parameters that position at least one tooth outside an associated dental arch may be within a range of safety limits for each position parameter. Furthermore, the range of safety limits may include a maximum difference between position parameters for a planned tooth position outside the associated dental arch and the desired final position of the patient's teeth.

The execution of the instructions for determining the difference between the desired final position of the patient's teeth and the predicted final position of the patient's teeth may include summing the difference between a position of each tooth in the desired final position of the patient's teeth and an associated tooth in the predicted final position of the patient's teeth. In some cases, determining the difference between the desired final position of the patient's teeth and the predicted final position of the patient's teeth may include summing the difference in each of six independent axes of motion between each tooth in the desired final position of the patient's teeth and an associated tooth in the predicted final position of the patient's teeth. Execution of the instructions may further cause the device to preferentially weighting a position of particular teeth included in the summed difference between a position of each tooth in the desired final position of the patient's teeth and an associated tooth in the predicted final position of the patient's teeth. In some cases, the weights of the positions of particular teeth may be determined based on a treatment target.

The execution of the instructions for determining the predicted tooth movement may include updating the predicted tooth movement based on tooth movement data from a previous dental treatment of the patient. In some examples, the predicted tooth movement may be based on patient age, patient gender, treatment type, or a combination thereof. In some other examples, the historic tooth movement data may include desired tooth movement data and achieved tooth movement data for a plurality of patients. The execution of the instructions may cause the device to perform operations including displaying, on a user display, an image of the desired final position of the patient's teeth and an image of the predicted final position of the patient's teeth. The user display may be included in a mobile device, a laptop computer, or a tablet computer. In some cases, determining the predicted tooth movement may be further based on the desired tooth movement of the patient.

For example, described herein are methods of generating and/or modifying a dental treatment plan for a patient. These methods may include: selecting a trained prediction model (e.g., a ML agent, ML model, neural network, etc.) based on one or more patient characteristics (e.g., including demographic information, therapeutic problem, and/or cosmetic problem), wherein the trained prediction model is trained, for each individual tooth of a dental arch, to include multiple translational and rotational directions (e.g., six rotational and translational directions, such as buccal/lingual, mesial distal, and intrusion/extrusion) for the individual tooth as well as reaction forces (e.g., multiple translational and rotational directions, such as the same six rotational and translational directions, e.g., buccal/lingual, mesial distal, and intrusion/extrusion) on the individual tooth due to one or more teeth that are adjacent to the individual tooth; generating a set of predicted tooth positions from the set of initial patient tooth positions, the treatment plan, and the selected trained prediction model; comparing the set of predicted tooth positions to the set of target tooth positions (e.g., comparing each of the multiple translational and rotational directions, such as each of the six rotational and translational directions, buccal/lingual, mesial distal, and intrusion/extrusion) to determine a difference indicator; if the difference indicator is less than the threshold value, outputting an optimized treatment plan based on the treatment plan; if the difference indicator is at or greater than a threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and outputting an optimized treatment plan based on the modified treatment plan.

Any of these methods and apparatuses may initially be provided with a set of initial patient tooth positions, a treatment plan and/or a set of target tooth positions. The set of initial patient tooth positions may be configured in any manner that may be understood and manipulated by the method and apparatus. For example, the set of initial patient tooth positions may include a digital model and/or digital scan of the patient's teeth. The digital model may be any appropriate digital representation, and may be segmented, including segmented into individual teeth and/or gingiva. The digital model may be a mesh model or other appropriate model. Similarly, the set of target tooth positions may include a digital model of the patient's teeth in the desired final configuration. The set of target tooth positions may be automatically, manually and/or semi-automatically determined. In general, the set of target tooth positions may be established by the user (e.g., dental professional). In some examples the set of target tooth positions may be derived from instructions provided by the dental professional along with an impression and/or scan of the patient's teeth.

The patient characteristics may include demographic information, such as age, gender, etc. In some examples the patient characteristic may be limited to demographic information. Alternatively, in some examples the patient characteristics may include a description of a therapeutic problem specific to the patient, which may be identified by the user or may automatically be identified, such as deep bite, arch expansion, etc. In some examples the patient characteristic may be or may include a description of a cosmetic problem, which may be identified by the patient, the user, and/or automatically detected (e.g., tooth spacing, etc.).

For example, a method of generating or modifying a dental treatment plan for a patient having a set of initial patient tooth positions, a treatment plan and a set of target tooth positions, may include: selecting a trained prediction model, wherein the trained prediction model is trained, for each individual tooth of a dental arch, to include multiple translational and rotational directions for the individual tooth as well as reaction forces on the individual tooth due to one or more teeth that are adjacent to the individual tooth; generating a set of predicted tooth positions from the set of initial patient tooth positions, the treatment plan, and the selected trained prediction model; comparing the set of predicted tooth positions to the set of target tooth positions to determine a difference indicator; if the difference indicator is at or greater than a threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and outputting an optimized treatment plan based on the modified treatment plan.

A method of generating or modifying a dental treatment plan for a patient having a set of initial patient tooth positions, a treatment plan and a set of target tooth positions, may include: selecting a trained prediction model based on one or more patient characteristic, wherein the trained prediction model is trained, for each individual tooth of a dental arch, to include a linear regression for each of multiple translational and rotational directions for the individual tooth as well as for multiple translational and rotational directions of one or more teeth that are adjacent to the individual tooth; generating a set of predicted tooth positions from the set of initial patient tooth positions, the treatment plan, and the selected trained prediction model; comparing the set of predicted tooth positions to the set of target tooth positions for each of the multiple translational and rotational directions to determine a difference indicator; if the difference indicator is less than a threshold value, outputting an optimized treatment plan based on the treatment plan; if the difference indicator is at or greater than the threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and outputting an optimized treatment plan based on the modified treatment plan.

In any of these methods and apparatuses, the trained prediction model may be trained, for each individual tooth of a dental arch, to include multiple translational and rotational directions for the individual tooth as well as reaction forces on the individual tooth due to one or more teeth that are adjacent to the individual tooth. For example, trained prediction model may include (e.g., may consider, as part of the training and linear regression) two or more teeth that are adjacent to the individual tooth (e.g., three or more teeth, four or more teeth, five or more teeth, six or more teeth, etc.). The adjacent teeth included may be symmetrically arranged adjacent to the individual tooth (e.g., on either side of the individual tooth) or asymmetrically arranged (e.g., only on one side or more on one side than the other, such as two on one side, one on the other side). The sides of adjacent teeth may refer to more anterior/posterior.

As mentioned, any of these methods may include generating a set of dental appliances based on the modified treatment plan. For example, the methods may include providing instructions for fabricating the dental appliances (aligners, including polymeric aligners) of a treatment plan. In some examples the methods may include fabricating the dental appliances, including fabricating by 3D printing.

Any of these methods may include selecting the trained prediction model by selecting a trained neural network from a library of trained neural networks indexed by patient characteristics. The library may be maintained in a remote database accessed by the local processor performing the method and/or may be remotely maintained. The trained neural networks may be updated and or modified to include additional patient data in an ongoing manner.

As mentioned, any of these methods and apparatuses may include selecting the trained prediction model by selecting the trained prediction model based on one or more patient characteristics including: the patient's age, the patient's gender, a therapeutic problem of the patient, and/or a cosmetic concern of the patient. For example, the library of trained neural networks may be specific to patient age ranges (e.g., less than 18, 18-45, 45 and older, etc.) and/or gender (male/female). Selecting the trained prediction model may include selecting a trained prediction model that is trained, for each individual tooth of a dental arch (or both sets of dental arches), using a linear regression model for each of the multiple translational and rotational directions. For example, the trained prediction model may be trained using a Huber linear regression model for each of the multiple translational and rotational directions.

As described above, the trained prediction models may be particularly effective because they use both individual tooth movements (translation and/or rotation) in multiple directions, such as buccal/lingual, mesial distal, and intrusion/extrusion; in addition, the trained prediction models include the reaction forces on the individual teeth from one or more adjacent teeth, such as the tooth movements (translation and/or rotation) of the adjacently flanking teeth when considering the individual teeth. Thus, the trained prediction model may consider three sets of six degrees of freedom for each tooth in the set, for a total of 18 parameters (e.g., six movements for the specified tooth and potential six movements for each of two adjacent teeth).

For example, selecting the trained prediction model may include selecting a trained prediction model that is trained for each individual tooth of the dental arch, to include multiple translational and rotational directions including six rotational and translational directions. The six rotational and translational directions may include: buccal/lingual, mesial distal, and intrusion/extrusion. Selecting the trained prediction model may include selecting a trained prediction model that is trained for each individual tooth of the dental arch, to include multiple translational and rotational directions for the reaction forces. As mentioned, the multiple translational and rotational directions the reaction forces may include six rotational and translational directions including: buccal/lingual, mesial distal, and intrusion/extrusion.

In any of these methods and apparatuses, comparing the set of predicted tooth positions to the set of target tooth positions may include determining a difference for each of the multiple translational and rotational directions for each tooth and combining the differences for each of the multiple translational and rotational directions for each tooth to determine the difference indicator. For example, comparing the set of predicted tooth positions to the set of target tooth positions may comprise determining a difference for each of the multiple translational and rotational directions for each tooth, weighting all or some of the differences of the multiple translational and rotational directions for each tooth, and combining the weighted and any unweighted differences for each of the multiple translational and rotational directions for each tooth to determine the difference indicator. For example, any of these methods may include receiving one or more weighting values specific to a clinician associated with the patient, wherein the one or more weighting values are used for weighting. Each degree of freedom (e.g., buccal/lingual, mesial distal, and intrusion/extrusion) may be weighted for each tooth. For example, if 14 teeth are present (e.g., in an upper arch), each with six degrees of freedom, then 84 weighting values may be used (some of which may be set to neutral, e.g., 1, or essentially unweighted). The methods and apparatuses described herein may include adding the differences of each tooth's degree freedom, some or all of which may be weighted, to form the difference indicator. Thus, the difference indicator may be weighted by weighting each of the six rotational and translational directions, e.g., based on physician preferences for all or some of the six rotational and translational directions of each tooth. As used herein, “unweighting” may be weighted with a weighting value of 1.

Also described herein are apparatuses, including one or more non-transitory computer-readable storage medium comprising instructions that, when executed by one or more processors of a device, cause the device to perform operations comprising: selecting a trained prediction model based on one or more patient characteristic, wherein the trained prediction model is trained, for each individual tooth of a dental arch, to include multiple translational and rotational directions for the individual tooth as well as reaction forces on the individual tooth due to one or more teeth that are adjacent to the individual tooth; generating a set of predicted tooth positions from a set of initial patient tooth positions, a treatment plan, and the selected trained prediction model; comparing the set of predicted tooth positions to the set of target tooth positions to determine a difference indicator; if the difference indicator is at or greater than a threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and outputting an optimized treatment plan based on the modified treatment plan.

For example, a non-transitory computer-readable storage medium comprising instructions that, when executed by one or more processors of a device, cause the device to perform operations comprising: selecting a trained prediction model based on one or more patient characteristic, wherein the trained prediction model is trained, for each individual tooth of a dental arch, to include a linear regression for each of multiple translational and rotational directions for the individual tooth as well as for multiple translational and rotational directions of one or more teeth that are adjacent to the individual tooth; generating a set of predicted tooth positions from a set of initial patient tooth positions, a treatment plan, and the selected trained prediction model; comparing the set of predicted tooth positions to the set of target tooth positions for each of the multiple translational and rotational directions to determine a difference indicator; if the difference indicator is less than a threshold value, outputting an optimized treatment plan based on the treatment plan; if the difference indicator is at or greater than the threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and outputting an optimized treatment plan based on the modified treatment plan.

All of the methods and apparatuses described herein, in any combination, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the methods and apparatuses described herein will be obtained by reference to the following detailed description that sets forth illustrative embodiments, and the accompanying drawings of which:

FIG. 1A schematically illustrates the steps of forming a treatment plan.

FIG. 1B schematically illustrates a method of optimizing a treatment plan as described herein.

FIG. 2 illustrates one example of a data structure for an optimization system/subsystem as described herein.

FIG. 3A shows one example of a schematic of a trained prediction model (e.g., a linear regression model).

FIG. 3B illustrates the use of multiple degrees of freedom for each tooth (tooth of prediction) including the use of any adjacent teeth when training a trained prediction model.

FIG. 4 shows a frontal view of example dental arches.

FIG. 5 shows an example block diagram depicting one example of data flow for determining a trained prediction model for predicting tooth movement.

FIG. 6 is a flowchart depicting an example of an operation of generating dental appliances that may be used to implement a treatment plan for a patient.

FIGS. 7A-7B show simplified diagrams of a dental arch depicting over-treatment.

FIG. 8 shows a block diagram of device that may be one example of a device that may be configured to perform any of the operations described herein.

FIG. 9 is a graph showing a comparison between ideal, optimized constrained, and optimized un-constrained (as well as upper and lower safety limits) for one example of a treatment plan as described herein.

FIGS. 10A-10B illustrate example overlays showing the final positions of a teeth in a constrained (FIG. 10A) and unconstrained (FIG. 10B).

FIGS. 11A-11B illustrate examples of overlays showing the final position of teeth when weighting either translation more than rotation (FIG. 11A) or rotation more than translation (FIG. 11B).

### DETAILED DESCRIPTION

The methods and apparatuses described herein may include a trained prediction model in that may be selected based on one or more patient characteristics and used to predict a patient's tooth movement for a proposed treatment plan. This information may be used to optimize the treatment plan by minimizing the difference between the predicted tooth position at the end of treatment and the target tooth position at the end of treatment (or at any point during the treatment). For example, the trained prediction models may be regression models that may be selected based on demographical info (ex. age, gender, etc.) and the treatment progress (extracted from progress scan) to predict the tooth movement. These method and apparatuses (e.g., software) may therefore optimize the treatment planning, resulting in a significantly higher aligner therapy efficacy.

As will be described in greater detail herein, these methods may also incorporate safety limits for tooth movement in the optimization process for treatment planning, and may provide extremely fast predictions that are easy to interpret and robust when adapting in new datasets.

For example, the methods described herein may improve the treatment planning for dental aligner therapy by improving the efficacy of treatment plans. Accurate and rapid predictions of patient tooth position may allow optimization of treatment plans that take individual difference between patients (and treatment providers) into account. For example, the methods described herein may compute an optimal treatment plan by utilizing a customized tooth movement prediction model to provide a constrained optimization method.

The trained prediction models described herein may use a linear regression model to predict tooth movement given a treatment plan, using one or more patient characteristic (e.g., demographic information such as age, gender, etc.). Based on the prediction of the tooth movement, the methods and apparatuses described herein may utilize a constrained optimization technique to design an optimal treatment plan, resulting in a higher aligner therapy efficacy.

To enhance the safety for treatment planning, this optimization process may allow users (e.g., dental professionals, such as orthodontist, dentists, doctors, technicians, etc.) to set weights (e.g., weighting coefficients) to boost or constrain the movements of a prescribed tooth set. This provides a closed-form solution which enables a low-cost, real-time processing for the treatment optimization.

When a new patient first starts, a scan of the patient's teeth may be made, and the dental professional may prescribe a treatment, including indicating a target position for the teeth (desired tooth position). The user and/or patient may provide patient characteristic information, including, e.g., demographic information. The user may also indicate clinical concerns (e.g., overbite, deep bite, arch expansion, etc.). The user and/or patient may indicate cosmetic concerns (e.g., alignment of particular teeth, gaps between teeth, etc.).

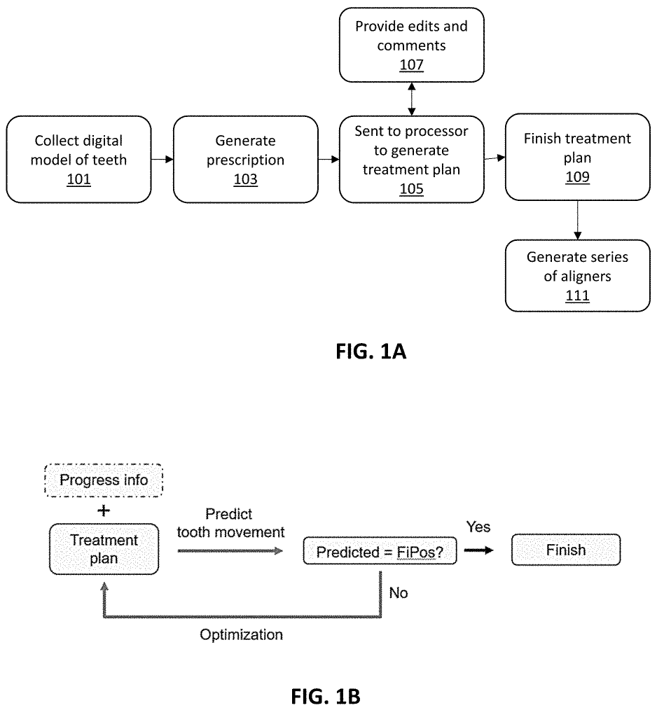

FIG. 1A shows a workflow for a traditional design of a treatment plan. In this example, the patient's teeth are digitally modeled, e.g., by scanning the teeth direction (e.g., using an intraoral scanner) and/or by scanning an impression of the teeth, etc. 101. The user may then generate a prescription, e.g., indicating the treatment targets and/or goals, as well as instructions on the treatment steps (e.g., the use of attachments, interproximal reductions, number of stages, etc.), 103 and this information may be transmitted to a processor that may generate a treatment plan 105. For example, the treatment plan may be generated by one or more modules (e.g., “FiPos”) that generates the final position. This target final position (“target position”) may be reviewed and iterated by the user and manually, automatically or semi-automatically modified 107. For example, the user may provide edits on the final positions and/or treatment steps. Once this process is completed, the final treatment plan may be generated 109 and, after final approval/review, a series of treatment aligners may be generated.

FIG. 1B illustrates and example of a method for modifying and/or optimizing the treatment plan as described herein. In this example, an initial treatment plan (e.g., from or at step 105 or 109 in FIG. 1A) may be optimized as described herein, for example, by using the initial patient tooth positions, the treatment plan and target tooth positions along with a regression model. The regression model may be used with the patient characteristic (e.g., demographical info such as age, gender etc., and/or clinical and/or cosmetic concerns) to select the regression model. The regression model may be referred to as the trained prediction mode, and may predict the tooth movement at the end of the treatment.

The methods or apparatus may also include checking whether the predicted tooth movements match with the desired tooth movement (i.e. FiPos) that users set. If there is a mismatch between the predicted tooth movement (e.g., predicted tooth position) and the desired tooth movement (e.g., target tooth position), the method or apparatus may modify the treatment plan with the optimization module, and may predict the tooth movement again based on the updated treatment plan. These optimization iterations may continue until the prediction of the tooth movements match with the prescribed target tooth movements (or target tooth positions, as generated by “FiPos”). Once the optimization iterations are completed, the method or apparatus may generate a series of aligners based on the optimized treatment plan.

For existing patients, the method or apparatus may leverage the scans from the patient's previous treatment (e.g., progress info) to account for the individual differences, for example, in wearing habits, bone physiology etc., into the regression model, and may further improve the tooth movement prediction. This is illustrated in FIG. 1B (“Progress info”). For example, to design the treatment plan for an existing patient, the method or apparatus may first extract the progress info from the patient (e.g., planned/achieved tooth movement from previous treatments), and may then utilize the progress info along with the current treatment plan and the one or more patient characteristic(s) (e.g., demographical info such as age, gender etc., and/or clinical concerns, and/or cosmetic concerns) as inputs to the regression model to predict the tooth movement at the end of the treatment. The method or apparatus may then follow the same optimization process described above, generating a predicted tooth movement (or equivalently in this context, tooth position), and determining a difference indicator between the two for each tooth of the dental arch in the multiple translational/rotational movements (e.g., buccal/lingual, mesial/distal, and intrusion/extrusion movements).

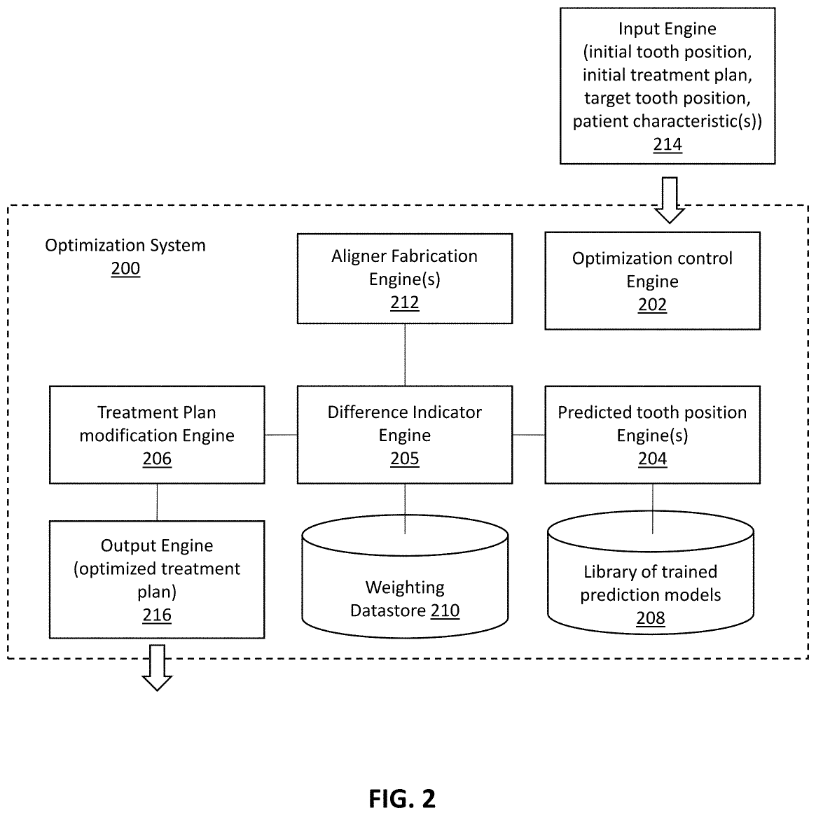

FIG. 2 is a diagram showing an example of an optimization system 200; the optimization system may be incorporated into a portion of another system (e.g., a general treatment planning system) and may therefore also be referred to as an optimization sub-system. In any of the method and apparatuses described herein the optimization system/sub-system may be invoked by a user control, such as a tab, button, etc., as part of treatment planning system, or may be separately invoked.

In FIG. 2, the optimization system 200 may include a plurality of engines and datastores. A computer system can be implemented as an engine, as part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The optimization system/sub-system 200 may include or be part of a computer-readable medium, and may include an input engine 214 (e.g., providing and/or allowing access to the patient's initial tooth position, initial treatment plan, target tooth position, and/or patient characteristic(s). The optimization system/subsystem may include an optimization control engine 202 that may control operation between the various engines of the optimization system/subsystem. In some examples the optimization system may include a memory, register or datastore string all or some of the patient's initial tooth position, initial treatment plan, target tooth position, and/or patient characteristic(s). The optimization system/sub-system may also include a predicted tooth position engine 204 that may select a trained prediction model from the library of trained prediction models 208 based on one or more patient characteristic and may generate a set of predicted tooth positions from the set of initial patient tooth positions, the treatment plan, and the selected trained prediction model. The set of predicted tooth positions may also be stored in a memory, register and/or datastore. The optimization system/subsystem may also include a difference indicator engine 205 that compares the set of predicted tooth positions to the set of target tooth positions for each of the multiple translational and rotational directions to determine a difference indicator. The difference indicator engine may also weight all or some of the multiple translational and rotational directions for each tooth before they are combined (after any weighting is applied, e.g., using the weighting datastore 210). The weighted and any unweighted differences for each of the multiple translational and rotational directions for each tooth to determine the difference indicator. The optimization control engine may then determine if the difference indicator is at or above a threshold indicating or is below a threshold. If the optimization control is below the threshold, then the treatment plan modification engine 206 may be evoked to iteratively modify the current treatment plan, apply the predicted tooth position engine using the modified treatment plan 204 and the difference indicator engine 205 until the difference indicator is below the threshold. Once the difference indicator is below the threshold (or after a completion criterion is met, such as a limit on the number of iterations), the optimization system may either or both fabricate an aligner or series of aligners using the aligner fabrication engine 212, and/or evoke the output engine 216 to output the optimized treatment plan.

One or more of the engines of the optimization system/subsystem 200 may be coupled to one another (e.g., through the example couplings shown in FIG. 2) or to modules/engines not explicitly shown in FIG. 2. The computer-readable medium may include any computer-readable medium, including without limitation a bus, a wired network, a wireless network, or some combination thereof.

The aligner fabrication engine(s) 212 may implement one or more automated agents configured to fabricate an aligner. Examples of an aligner are described in detail in U.S. Pat. No. 5,975,893, and in published PCT application WO 98/58596, which is herein incorporated by reference for all purposes. Systems of dental appliances employing technology described in U.S. Pat. No. 5,975,893 are commercially available from Align Technology, Inc., Santa Clara, Calif., under the tradename, Invisalign System. Throughout the description herein, the use of the terms “orthodontic aligner”, “aligner”, or “dental aligner” is synonymous with the use of the terms “appliance” and “dental appliance” in terms of dental applications. For purposes of clarity, embodiments are hereinafter described within the context of the use and application of appliances, and more specifically “dental appliances.” The aligner fabrication engine(s) 108 may be part of 3D printing systems, thermoforming systems, or some combination thereof.

In implementation, the methods and apparatuses described herein may include trained prediction models that are based on linear regression models for prediction of tooth movement/position. For example, a linear regression model to predict the tooth movement may include inputs including multiple translational and/or rotational directions for each tooth. For example, the trained prediction model may include all six degrees of freedom (DOF) for planned movements from the previous treatment (e.g., for existing patients), and/or all six DOF of progress movement in the previous treatment (e.g., for existing patients), and/or all six DOF of planned movement in the current treatment, as well as the one or more patient characteristics (e.g., patient demographical info such as age, gender etc.).

When predicting the movement of a tooth the apparatus or method may also use the information from adjacent teeth as well as the tooth being examined. For example, in a 3-tooth model the total inputs to predict the predicted movement for tooth #5 are: 3 (tooth #4, 5, 6)*6 (dof)*3 (tooth movement info a. to c.)=54 features.

One goal of this optimization methods and apparatuses described herein is to find the optimal planned tooth movement for a treatment, so that the predicted tooth movement matches with the prescribed desired movement (e.g., FiPos) as much as possible. Moreover, this optimization process may allow users to set weighting coefficients to boost or constrain the movements of a specific tooth set.

This optimization problem can be formulated as:

$$Minimize : {weighted error}^{} = {weight}^{} \cdot ( {predicted movement}^{} - {desired movement}^{} ) \qquad [ Equation 1 ]$$

In this expression, i=1, 2, . . . , 6n denotes the total components (6 dof*n tooth) of tooth movements. Since the predicted tooth movement may be computed by a linear regression model, the method or apparatus can extract the regression coefficients and manually derive the closed form solution for this optimization problem. In this case the treatment optimization process can be done in real time.

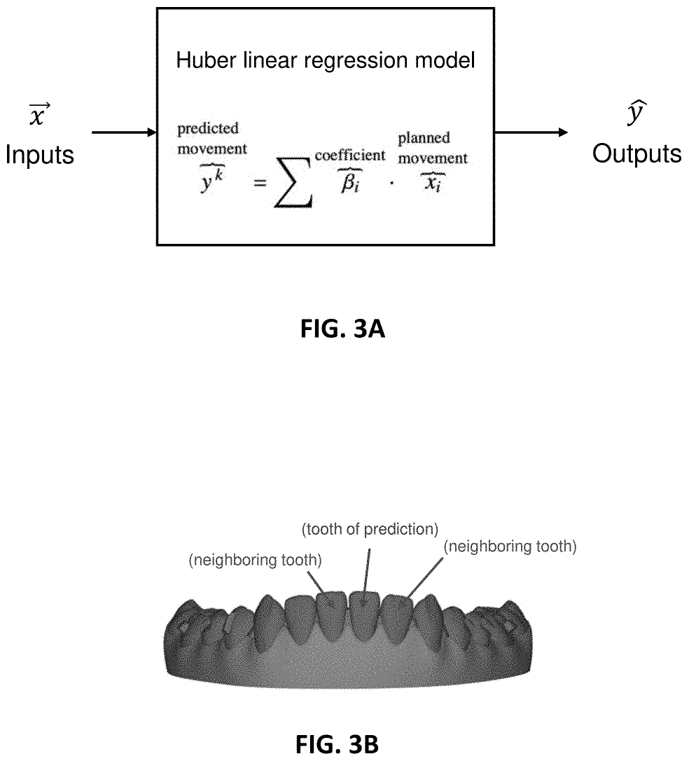

FIG. 3A schematically illustrates an example of a trained prediction model (e.g., a linear regression model) based on a Huber linear regression model. In this example the inputs may include planed tooth movements values. In operation, for each tooth in the dental arch examined, the trained prediction model may consider both the degrees of freedom of the tooth itself as well as the adjacent one or more teeth that may exert reaction forces on the tooth. Thus, the inputs may be planned tooth movement values (e.g., 3 teeth*6 directions), planned movement (e.g., 3 translations+3 rotations), and neighboring planned movement. The outputs may include the prediction of tooth movement values (in each of six directions). The three teeth considered for each individual tooth prediction determination are shown in FIG. 3B; the central tooth is the tooth of prediction, which is flanked by two neighboring teeth. In some cases only a single (or no) neighboring teeth may be present. The use of neighboring teeth may allow for the effect of the reaction force(s) applied on the tooth of prediction by the neighboring teeth. For each of these three teeth both translation and rotation may be considered, which may allow for the effects of tipping.

A method and a non-transitory computer-readable medium for generating dental appliance data may include operations for predicting tooth movement based on an analysis of a group of historical patient data. In some cases, a predicted tooth position may be based on not only a patient's initial and desired tooth positions, but also on interactions that a target tooth may have with adjacent teeth. In some cases, the historical patient data may be machine analyzed (e.g., computer analyzed) to determine a prediction model that takes into account influences from one or more adjacent teeth on a target tooth.

The prediction model may be used to determine a patient's predicted final tooth position using a patient's treatment plan. The treatment plan may include a patient's initial and desired tooth positions. Thus, the prediction model may determine a patient's initial tooth positions and desired tooth movements from the patient's treatment plan and predict the patient's final tooth position. If the final predicted tooth position is not sufficiently close to the desired final tooth position (for example, the final predicted tooth position is not within a predetermined measure of error from the desired final tooth position), then a clinician may modify the patient's treatment plan and iterate through the prediction model. On the other hand, if the final predicted tooth position is sufficiently close to the desired final tooth position, then one or more dental appliances (dental aligners) may be fabricated to implement the treatment plan.

In some cases, a patient's own dental history regarding tooth movement may be used to predict the patient's response to further dental treatment. For example, information associated with the patient's tooth movement from a previous dental treatment may be provided to the prediction model to help determine an updated predicted tooth position resulting from implementing a treatment plan.

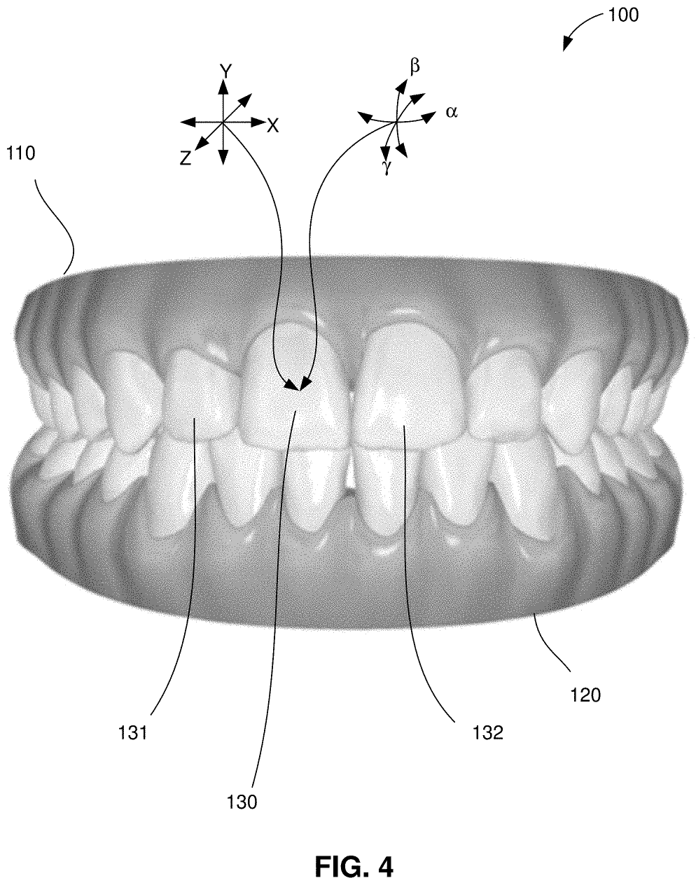

FIG. 4 shows a frontal view of example dental arches 100, in accordance with some embodiments. The dental arches 100 may include an upper dental arch 110 and a lower dental arch 120. A typical patient may have fourteen teeth on the upper dental arch 110 and fourteen teeth on the lower dental arch 120. The any one tooth may be described as having six degrees of freedom. In other words, any one tooth may have tooth motion described as a translation within six independent and separate axes.

For example, movement of tooth 130 in the upper dental arch 110 may be described as moving a distance (e.g., any feasible amount) in six independent axes. Three of the axes of movement may be linear axes associated with traditional linear movement in space. For example, movement may be along buccal/lingual, mesial/distal, and intrusion/extrusion axes (referred to herein as x, y, and z axes). Three additional rotational axes of movement may be associated with a rotational movement in space (referred to herein as α, β, and γ axes). An example of rotational movement may be sometimes referred to a pitch, yaw, and roll. As depicted in FIG. 4, movement of tooth 130 may be described as a movement in the six axes (x, y, z, α, β, and γ axes). Any amount of movement within any of these six axes may be referred to as a parameter. Thus, for any one dental arch, the movement of 14 teeth may be described with 84 parameters (14 teeth times six parameters).

In some cases, an optimized tooth movement model may consider influences of adjacent teeth on the movement of a target tooth. For example, biomechanical interactions of adjacent teeth 131 and 132 with tooth 130 may be used to predict the tooth movement of tooth 130. Since the movement of any one tooth may be described with six parameters, the prediction of the movement of any one tooth may consider up to 18 parameters (three teeth times six parameters).

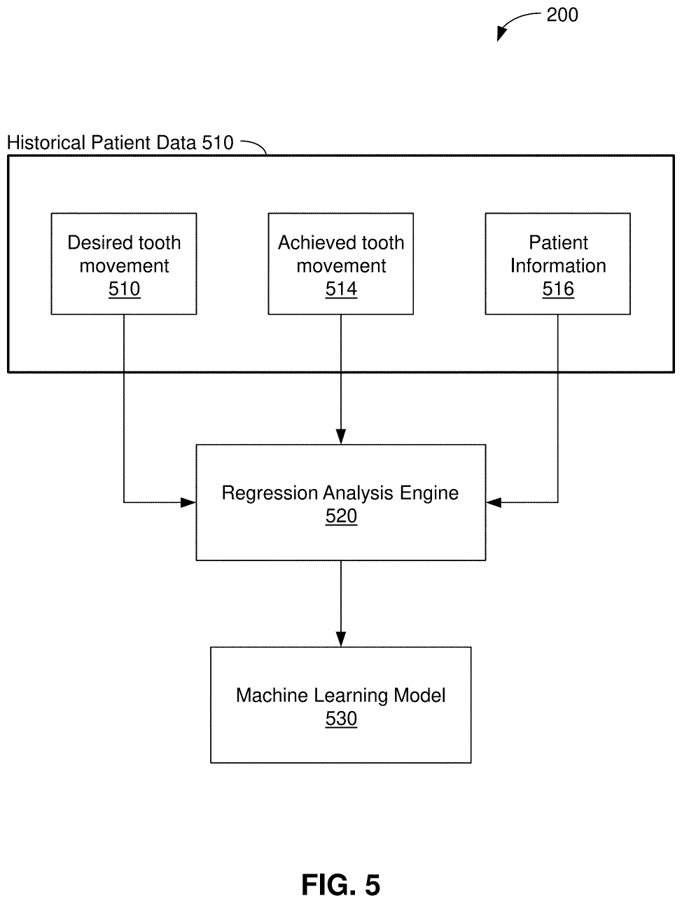

FIG. 5 shows an example block diagram 500 depicting one example of a data flow for determining a trained prediction model (e.g., a machine learning model) for predicting tooth movement. As shown, historical patient data 510 is used by a regression analysis engine 520 to generate a trained prediction model 530.

Dental and/or orthodontic procedures may, for example, implement a treatment plan that involves the movement of one or more of the patient's teeth. In some cases, a model for predicting tooth movement may be based on an analysis of historical patient data 510 that includes information regarding the tooth movement of a number of patients referenced to a number of treatment plans. In some cases, the historical patient data 510 may include treatment data associated with several (thousands, tens of thousands, or more) patients that have undergone procedures that involve the movement of teeth.

The historical patient data 510 may include desired tooth movement data 512, achieved tooth movement data 514, and patient information 516 to describe historic tooth movement referenced to patient treatment plans. The desired tooth movement data 512 and the achieved tooth movement data 514 may collectively be referred to as historic patient tooth movement data. In some cases, the desired tooth movement data 512 may correspond to a desired treatment (e.g., a patient's treatment plan) to move a patient's teeth and describe tooth movement in six axes (x, y, z, α, β, and γ axes). Thus, for each patient, the desired tooth movement data 512 may include six movement parameters for each tooth in the patient's upper and lower dental arches.

The achieved tooth movement data 514 may describe actual tooth movement that was achieved in response to the patient's treatment plan. Thus, for each patient, the achieved tooth movement data 514 may include six movement parameters for each of the patient's teeth. These movement parameters may differ from the movement parameters in the desired tooth movement data 512 as these parameters reflect realized (instead of desired) tooth movement. In some cases, the realized tooth movement may be less than the desired tooth movement. Further, since the achieved tooth movement data 514 may include movement data of all the patient's teeth, inferences may be made regarding the influence of adjacent (e.g., neighboring) teeth on the motion of a target tooth. These inferences may be used to determine the trained prediction model 530.

The patient information 516 may include additional information associated with the patient as related to the historic patient tooth movement data (the desired tooth movement data 512 and the achieved tooth movement data 514). In some cases, the patient information 516 may include patient age and/or gender information. For example, patients younger than 18 years old may respond to a treatment plan differently than patients 18 years old or older. Similarly, male patients may respond to a treatment plan differently than female patients. In some other cases, the patient information 516 may include a treatment category. Example treatment categories may include treatment types such as deep bite treatment, arch expansion treatment as well as any other feasible treatment type.

The patient information 516 may be used determine one or more trained prediction models 530. For example, the desired tooth movement data 512 and the achieved tooth movement data 514 may be grouped together based on patient information 516. In this manner, a trained prediction model may be generated for different categories or groupings of patient information 516. Any number of categories or groupings are possible. For example, a trained prediction model 530 may predict tooth movement related to deep bite treatment for patients older than 18 years old. In another example, a trained prediction model 530 may predict tooth movement related to arch expansion treatment for patients that are younger than 18 years old.

The historical patient data 510 is provided to the regression analysis engine 520. The regression analysis engine 520 may perform one or more types of regression analysis on the historical patient data to determine the trained prediction model 530. For example, the regression analysis engine 520 may perform linear regression using the desired tooth movement data 512, the achieved tooth movement data 514, and the patient information 516 to determine a prediction model that predicts tooth movement for a patient. In some cases, the regression analysis engine 520 may perform linear regression analysis on the desired tooth movement data 512 and the achieved tooth movement data 514 group according to patient information 516. For example, the desired tooth movement data 512 and the achieved tooth movement data 514 for deep bite treatments may be grouped by patient information 516 and analyzed to determine a trained prediction model 530 to predict tooth movements associated with deep bite treatments.

Furthermore, any determined trained prediction model 530 may be further sub-categorized based on other patient information 516. For example, a first trained prediction model 530 for deep bite treatments may be determined for patients younger than 18 years old and a second trained prediction model 530 for deep bite treatments may be determined for patients 18 years old or older. Although only deep bite treatments and patient age are mentioned above, separate trained prediction models 530 may be determined for any feasible group or groupings of patient information 516.

The regression analysis engine 520 may determine a trained prediction model 530 that predicts tooth movement by examining the desired and achieved tooth movement of a target tooth while also examining the desired and/or achieved tooth movement of teeth adjacent to the target tooth. Thus, a predicted tooth movement for a target tooth may be determined based on biomechanical interactions between two adjacent teeth. For example, although a target tooth may be moved in response to a treatment plan, a tooth to the right and a tooth to the left of the target tooth may cause tipping and/or rotation to the target tooth. Thus, 18 tooth movement parameters may be used to predict movement of any one target tooth. Therefore, using movement information from adjacent teeth may allow the trained prediction model 530 to include tooth movement due to biomechanical interaction with adjacent teeth.

Although described above as using two adjacent teeth to predict the movement of a target tooth, any number of adjacent teeth may be used. For example, instead of just two adjacent teeth, four adjacent teeth may be used to determine tooth movement. That is, two teeth to the right of the target tooth and two teeth to the left of the target tooth may be used to predict the movement of the target tooth. In some cases, different numbers of teeth may be used to either side of the target tooth. For example, one tooth to the left of the target tooth and two teeth to the right of the target tooth may be used to predict the movement of the target tooth.

In some variations, the regression analysis engine 520 may perform linear regression analysis on a target tooth and one or more adjacent teeth to determine a trained prediction model 530 that predicts tooth movement. In this manner, biomechanical interaction between teeth may be used to predict tooth movement. In some cases, results of the linear regression may be expressed as a coefficient that may be applied to a desired position of a target tooth.

For example, for a given treatment plan, a target tooth may have a desired tooth movement that may be expressed as:

X_{D}=(x₁,x₂,x₃,x₄,x₅,x₆) [equation 2]

### Where

X_{D} is the desired movement of a target tooth; and
x, is measure of the movement within each of the six axes described above (e.g., x, y, z, α, β, and γ axes).

The desired tooth movement may be included within the desired tooth movement data 512. In some cases, the movement x, may be expressed in millimeters, however any feasible measurement system may be used. In a similar manner, the achieved tooth movement of a target tooth and adjacent teeth may be expressed as:

X_{T}=(x_{T1},x_{T2},x_{T3},x_{T4},x_{T5},x_{T6}) [equation 3]

X_{A}=(x_{A1},x_{A2},x_{A3},x_{A4},x_{A5},x_{A6}) [equation 4]

X_{B}=(x_{B1},x_{B2},x_{B3},x_{B4},x_{B5},x_{B6}) [equation 5]

### Where

X_{T} is the achieved tooth movement of a target tooth; and
X_{A} and X_{B} are the achieved tooth movements of teeth adjacent to the target tooth.
   Note that X_{T}, X_{A}, and X_{B} as expressed in equations 3-5 indicate the achieved movement of the respective teeth within the six axes similar to equation 2.

Applying linear regression to the historical patient data 510 (in particular to historical patient data of achieved tooth movement expressed as in equations 3-5) may determine the trained prediction model 530 that predicts movement of one or more target teeth. In some cases, the trained prediction model 530 may be said to have been “trained” based on the historical patient data 510.

In some variations, the trained prediction model 530 may be expressed as coefficients that may be applied to the desired tooth movement of a target tooth based on a treatment plan. For example, if the desired tooth movement is expressed as shown in equation 2, then a predicted tooth movement of a target tooth T may be expressed as:

Y_{T}=(δ₁x₁,δ₂x₂,δ₃x₃,δ₄x₄,δ₅x₅,δ₆x₆). [equation 6]

### Where

Y_{T} is the predicted movement of a target tooth T;
xᵢ is a desired movement within the six axes described above (e.g., x, y, z, α, β, and γ axes); and
δᵢ is a coefficient used to scale a respective desired movement xᵢ.

A simplified expression for the predicted movement of the target teeth may be expressed as:

Yⱼ=(δⱼₖ)(xⱼₖ) [equation 7]

### Where

Yⱼ is the predicted position of a target tooth J;
xⱼ is a desired movement of tooth J that encompasses movement within the six axes;
δj are the coefficients for the associated desired movements of tooth J;
j is from 1 to N (N=number of teeth in upper and lower dental arches); and
k is from 1 to 6 for movement within each of the six axes.

As described above, linear regression may be applied to historical tooth movements of a target tooth and two or more adjacent teeth to determine the coefficients used to predict movement of a target tooth. In some variations, Huber linear regression may be used by the regression analysis engine 520 as an alternative to conventional linear regression. Huber linear regression is a well-known linear regression technique that may reject or provide less weight to outlier historical patient data 510. Although conventional linear regression and Huber linear regression are mentioned here, any other feasible regression techniques or procedures may be used. For example, logistic regression, ridge regression, lasso regression, polynomial regression, or Bayesian linear regression may be used.

In some variations, a patient's own historical data may be used to determine or modify a trained prediction model 530 that is customized for the patient. In other words, predicted tooth movements may be determined based on the patient's own dental history (e.g., a patient's previous dental treatment). For example, if a patient has had previous dental treatment moving one or more teeth, then the associated patient data regarding desired and/or achieved tooth movement associated with the previous dental treatment may be provided to the regression analysis engine 520. The regression analysis engine 520 may then generate a trained prediction model 530 incorporating the patient's own teeth movement history.

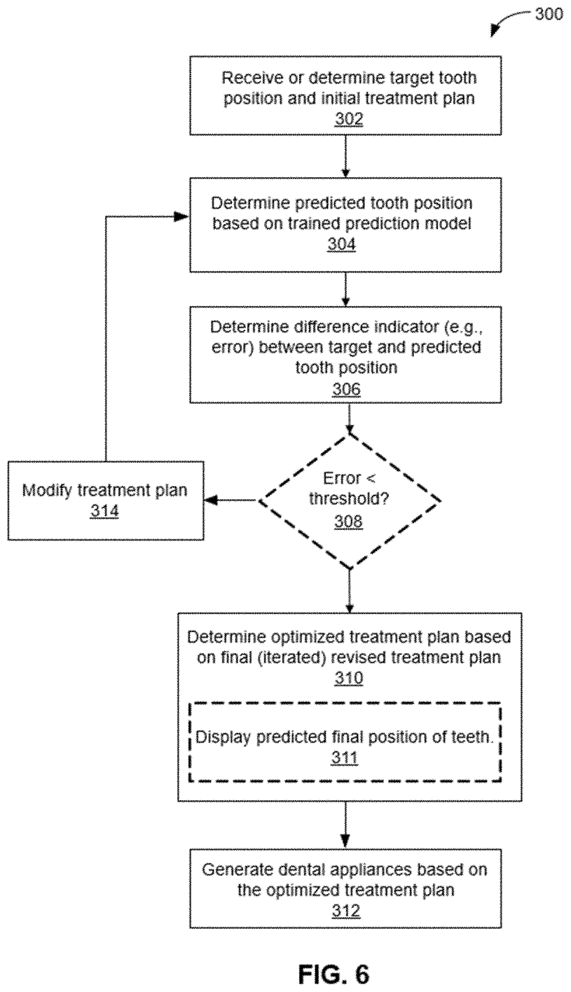

FIG. 6 is a flowchart depicting an example of an operation 300 of generating dental appliances that may be used to implement a treatment plan for a patient, in accordance with some embodiments. Some examples may perform the operations described herein with additional operations, fewer operations, operations in a different order, operations in parallel, and some operations differently. In some variations, a treatment plan may be modified (e.g., iterated) based on results of the predicted tooth movement.

The optimization operation 300 may begin in block 302 as a target tooth position (e.g., tooth movement) for the patient is provided and/or determined. The patient's target tooth movement may be determined as described above, by the user (e.g., dental professional), and may be used to generate an initial treatment plan. For example, a patient's treatment plan for palatal expansion, deep bite treatment, or the like may describe desired tooth movements for the patient's teeth that are intended to end in the target position (or close to the final position). In some cases, the desired movements for each of the patient's teeth may be expressed with parameters that indicate movement in six axes, such as described with respect to equation 2.

Next, a trained prediction model may be used to determine the patient's predicted tooth movement 304. The trained prediction model may be determined as described in conjunction with FIG. 5. Thus, a patient's predicted position may be determined using the treatment plan and/or the target tooth movement and the trained prediction model 230.

A difference indicator may be determined 304, indicating a difference between the target tooth position and the predicted tooth position. The difference between the target (or desired) tooth position of each tooth and the predicted tooth position of each tooth may be expressed as a total error E. In some embodiments, the total error E may be expressed as:

E=Σ_{T=1}^{N}(Y_{T}−X_{T})² [equation 8]

### Where

E is the total error;
Y_{T} is the predicted movement of tooth T;
X_{T} is the desired movement of tooth T; and
N is the number of teeth for the patient.

Note that the movement (e.g., predicted movement, target or desired movement) and position (e.g., predicted position, target or desired position) may be used equivalently in this description. The predicted position and desired position of each tooth T may be expressed as movement in six axes. In other words, the desired movement X_{D} may be expressed as equation 2 and the predicted movement Y_{T} of the corresponding tooth may be expressed as equation 6. Thus, the error between X_{D} and Y_{T} may be the sum of differences between of each of the tooth's associated parameters (e.g., the sum of the difference between the x axis parameters x₁ and δᵢx₁, the difference between y axis parameters x₂ and δ₂x₂, and so forth).

In some embodiments, a weighted error may be determined as an alternative to the total error of equation 8. The weighted error may place more importance or “weight” on the position of certain teeth. The weight may be added into equation 8 by introducing a weighting factor W_{T}. Thus, each tooth may have a unique weighting factor. In some variations, the weighting factor W_{T} may be between zero and one (0<=W_{T}<=1). The weighted error may be expressed below as:

E_{W}=E_{T=1}^{N}W_{T}(Y_{T}−X_{T})² [equation 9]

### Where

E_{W} is the weighted error;
W_{T} is the weighting factor of tooth T;
Y_{T} is the predicted movement of tooth T;
X_{T} is the desired movement of tooth T; and
N is the number of teeth for the patient.

The weighting factor W_{T} enables more weight (importance) to be placed on the movement error of certain teeth. To ease the determination of the weighted error E_{W}, the weighting factor W_{T} may be predetermined. In one example, the weighting factor W_{T} may be predetermined based on the treatment plan. For example, if the treatment plan is associated with a deep bite procedure, then the predetermined weighting factors W_{T} associated with correcting and/or addressing a deep bite may be used to emphasize the error (and therefore the movement) of particular teeth associated with deep bite treatment. Similarly, if the treatment plan is associated with palatal expansion, then predetermined weighting factors W_{T} associated with palatal expansion may be used to emphasize the error of particular teeth associated with palatal expansion.

In other embodiments, the predetermined weighting factors W_{T} may be associated with any feasible treatment or any other characteristic. For example, a predetermined weighting factor W_{T} may be associated with the age of the patient, the gender of the patient, or the like. In still other embodiments, the weighting factors may W_{T} be determined by a clinician.

In block 308, the determined difference indicator (e.g., error) may be compared to a threshold. Since the error measures a difference between a predicted and target position/amount of tooth movement, the amount of error can provide a measure of accuracy of an implemented treatment plan. The threshold may be an arbitrary number that may be clinically determined, and/or may be set by a dental professional (e.g., treating, supervising, or reviewing doctor, technician, specialist or the like) or determined through laboratory tests, bench tests, or simulations. In some variations, block 308 may be optional (as indicated by the dashed lines in FIG. 6). If operations for block 308 are optionally not performed, then the operation proceeds to block 310.

If the error (total error or weighted error) is less than a threshold, then the operation proceeds to block 310 where an optimized treatment plan is based on the last predicted final position of the patient's teeth. In some cases, the predicted and/or target final position may be displayed on a user's display 311. This operation may be optional. For example, the predicted final position of the patient's teeth may be displayed on a screen of a mobile device, a tablet computer, a laptop computer, a desktop computer, or any other feasible display device. In some cases, the predicted final position of the patient's teeth is displayed with an initial position of the patient's teeth. The initial and predicted images may be superimposed to highlight the changes in tooth position brought about by the treatment plan used to predict the final position of the patient's teeth.

Next, one or more (e.g., a series) of dental appliances may be generated 312 based on the modified treatment plan. In particular, block 312 may also include operations to generate dental appliance data used to manufacture/create dental appliances. In this manner, the treatment plan used to determine the predicted tooth movements may be implemented. In some variations, the dental appliances may be clear aligners that may be worn by the patient. In some embodiments, a plurality of dental appliances may be generated to implement the treatment plan though a number of stages. In some cases, the treatment plan may be divided into stages and a separate dental appliance may be used to implement each stage.

Returning to block 308, if the error is greater than (or in some cases equal to) the threshold, then operations proceed to block 314 where the treatment plan is revised so that the predicted tooth position may more closely align with the intended target final tooth position. In most cases, this means that the modified treatment plan is an overcorrection of the patient's teeth to allow the teeth to move more correctly towards the correct final position. As described below, the modification of the treatment plan may be made within limits or parameters preventing overcorrection beyond a reasonable threshold.

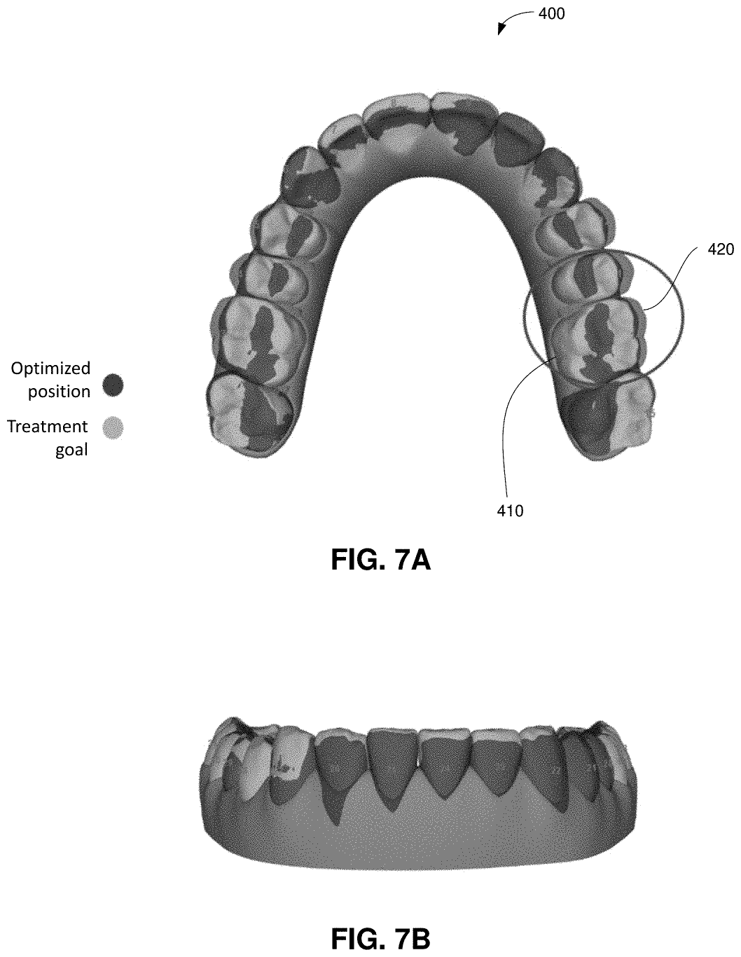

For example, in some cases, the treatment plan may be revised by modifying the apparent final position of one or more teeth. The modified apparent final position may be referred to as an over-treatment final position and is described in more detail with respect to FIGS. 7A-7B. After the treatment plan is revised, operations may return to block 304. In this manner, a new predicted tooth movement is determined and a new error calculated. The operations of blocks 304, 306, 308, and 314 may be iterated until the determined error is less than the threshold (block 308) or, in some cases, until a predetermined number of iterations has occurred (e.g., a completion criterion).

FIG. 7A is a simplified diagram of a dental arch 400 depicting over-treatment, in accordance with some embodiments. Although an upper dental arch is shown, over-treatment may be performed on any tooth in any dental arch.

Over-treatment may refer to a treatment protocol that may be applied to any tooth or groups of teeth included in a patient's treatment plan. During over-treatment, the apparent final position of one or more teeth is modified from their original or “true” desired final position to an over-treatment final position. In many cases, the over-treatment final position is not on or along the dental arch that includes the desired final position of the teeth specified in the patient's treatment plan.

For example, in the dental arch 400, tooth 410 is shown in a desired final position. In some cases, while implementing a treatment plan, a predicted final position of a tooth may not coincide or be near a desired final position. Some teeth may, in some cases, be subject to biomechanical forces from adjacent teeth that affect the movement of the tooth. In order to address these biomechanical forces, the desired final position may be modified to an over-treatment final position.

In some variations, the over-treatment final position is beyond the desired final position with respect to the dental arch. By implementing a treatment plan with one or more over-treatment positions, the movement of particular teeth may overcome some or all of the biomechanical forces exerted on a tooth by adjacent teeth.

The over-treatment final position may be constrained by one or more limits. These constraints may be referred to as safety constraints. Using over-treatment positions, without any constraints, may allow the over-treatment final position to drive a tooth to a dangerous and/or nonsensical position well beyond a desired dental arch. In some variations, the constraints may place a limit (sometimes referred to as a safety limit) on the over-treatment final position. Thus, the over-treatment final position may have a predefined distance limit with respect to the desired final position in any of the possible or feasible axes of movement. For example, the over-treatment final position of a tooth may be within one millimeter (mm) along any of the linear axes from the desired final position of the tooth. In another example, the over-treatment final position of a tooth may be within ten degrees on any of the rotation axes from the desired final position of the tooth. Any feasible predefined limit for any axis may be used.

Similar to as described above with respect to FIG. 6, after the over-treatment final position is determined, a trained prediction model may be used to determine an updated final position prediction. If the predicted results are still unsatisfactory (for example, the associated error is too large), then a new over-treatment position may be determined, and the trained prediction model again used to determine another updated final position prediction.

The error between the predicted final over-treatment position and the desired final tooth positions may be determined similar to as described with respect to equation 8 and 9 above. For example, using equation 9 the position of the tooth or teeth subject to over-treatment may be given more weight than other teeth when determining a weighted error.

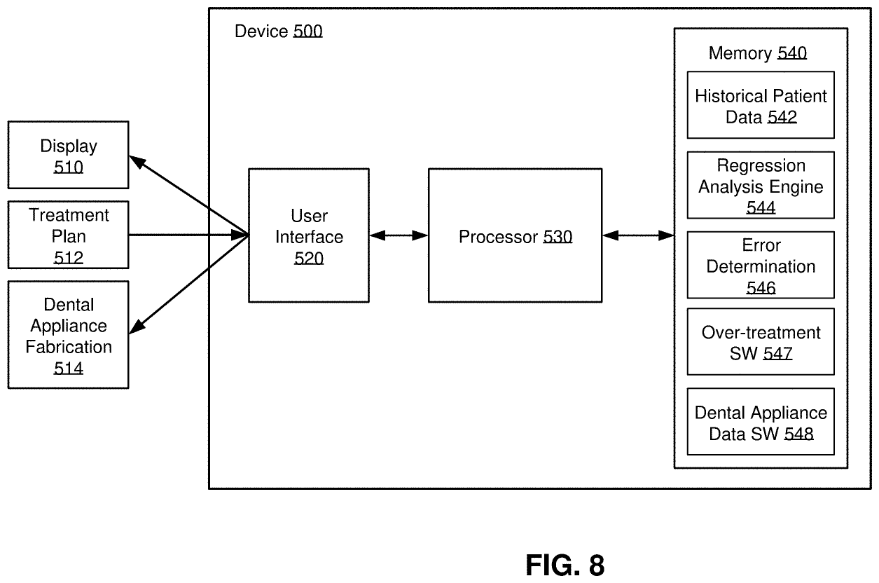

FIG. 8 shows a block diagram of device 500 that may be one example of a device that may be configured to perform any of the operations described herein. The device 500 may include a user interface 520, a processor 530, and a memory 540. The device 500 may be local (near) the user (clinician) that wants to determine dental appliance (e.g., dental aligner) data. In some variations, the device 500 may be remote (separate) from the user. For example, the device 500 may be implemented as a server or may be distributed on two or more servers or may be cloud (internet) based.

The user interface 520, which is coupled to the processor 530, may be used to interface with any device that receives or transmit data to and/or from the device 500. For example, the user interface 520 may be coupled to a display 510. The display 510 may show the user predicted, desired, and original tooth positions. The display 510 may be included on a mobile device such as a smart phone, a tablet computer, or laptop. The display 510 also may be included on devices that are not conventionally mobile such as a desktop computer or wall mounted display screen.

The user interface 520 may be coupled to a dental appliance fabrication unit 514. The dental appliance fabrication unit 514 may receive dental appliance data generated by the device 500 and, in turn, generate dental appliances. In some cases, the dental appliance data from the device 500 may be used to generate dental aligners, including clear dental aligners.

The user interface 520 may receive treatment plan data 512. For example, treatment plan data 512 may include a patient's initial and desired tooth positions. In some cases, the treatment plan data 512 may also include a patient's own historic data regarding previous dental treatment.

The processor 530, which is also coupled the memory 540, may be any one or more suitable processors capable of executing scripts or instructions of one or more software programs stored in the device 500 (such as within memory 540).

The memory 540 may include historical patient data 542. The historical patient data 542 may include desired and/or achieved tooth movement data for any number of patients that have undergone some dental treatment. The historical patient data 542 may also include other patient information including age and gender of the patients as well as the types of treatment the patient received. For example, the treatment type may be related to deep bite or palatal expansion treatments, although any treatment type is possible.

The memory 540 may also include a non-transitory computer-readable storage medium (e.g., one or more nonvolatile memory elements, such as EPROM, EEPROM, Flash memory, a hard drive, etc.) that may store the following software modules: a regression analysis engine 544 to determine a prediction model; an error determination module 546 to determine an error between a desired and a predicted tooth position; an over-treatment software (SW) module 547 to determine over-treatment tooth position values; and a dental appliance SW module 584 to generate dental appliance data.

Each software module, module, or engine includes program instructions that, when executed by the processor 530, may cause the device 500 to perform the corresponding function(s). Thus, the non-transitory computer-readable storage medium of memory 540 may include instructions for performing all or a portion of the operations described herein.

The processor 530 may execute the regression analysis engine 544 to determine/generate a prediction model to predict the tooth movement of a patient. For example, execution of the regression analysis engine 544 may perform a linear regression on the historical patient data 542 to determine a trained prediction model to predict tooth movement. In some variations, the linear regression may be a Huber linear regression. In some embodiments, the prediction model may be as expressed by equation 6 or equation 7, although the prediction model may be expressed or implemented using any feasible equation.

The processor 530 may execute the error determination module 546 to determine an error between a predicted final tooth position and a desired final tooth position. In some variations, execution of the error determination module 546 may include obtaining and providing a patient's treatment plan (included within treatment plan data 512) to the trained prediction model determined with the regression analysis engine 544 to generate the patient's predicted final tooth position. Error determination may be implemented as described above with respect to equations 8 and 9. In some variations, execution of the error determination module 546 may determine an error between a desired final tooth position and a revised predicted final tooth position. The revised final tooth position may be in response to iterations to reduce the determined error to less than a threshold. The revised final tooth position may also be in response to over-treatment tooth positions, for example provided by the over-treatment software module 547.

The processor 530 may execute the over-treatment software (SW) module 547 to determine over-treatment tooth positions for a patient. In some variations, execution of the over-treatment SW module 547 may determine over-treatment tooth positions that are within a predetermined limit (e.g., a safety limit). After the over-treatment tooth positions are determined, an associated revised treatment plan may be provided to the regression analysis engine 544 based on the over-treatment tooth positions. A new error associated with the revised treatment plan may be determined through execution of the error determination module 546.

The processor 530 may execute the dental appliance data SW module 548 to generate the dental appliance data that, in turn, may be used to generate dental appliances. The dental appliance data may correspond to a treatment plan associated with an error or weighted error that is less than a threshold. The treatment plan may be determined in conjunction with a predicted tooth position provided by a trained prediction model determined with the regression analysis engine 544. In some variations, the dental appliances may be dental aligners.

In some variations, the dental appliance data may be used to generate an image that may be displayed to the user. For example, an image of the predicted final tooth position may be displayed based on the dental appliance data. In some other variations, the predicted final tooth position may be superimposed with a patient's beginning or initial tooth position on a display device.

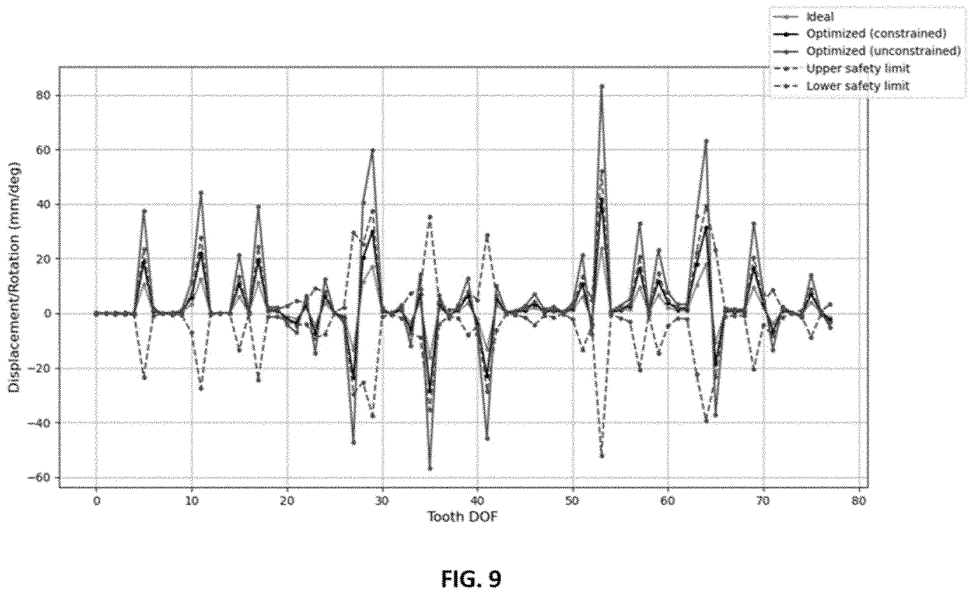

The methods described herein were validated using patient data. For example, to validate the model a test of clinical data (2000 arch expansion+2000 deep bites cases) in predicting the tooth movement in the primary treatment was tested. The R² score can reach 0.7-0.8. For example, a linear regression model for each axis of movement for a portion of a trained prediction model may be used. An optimal treatment plan designed by the optimization process described herein is shown in FIG. 9. In this plot the gray line denotes the original planned tooth movement, and the red line and black line denote the unconstrained and constrained optimized treatment plan, separately. The proposed model can be use in treatment optimization such as over treatment.

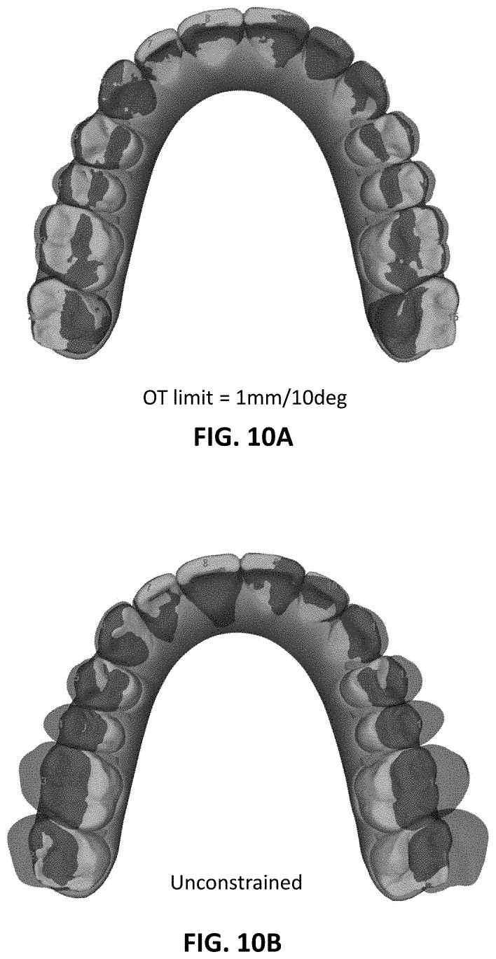

As mentioned above, in any of the methods and apparatuses described herein the modified treatment plans described herein, which may be modified in order to achieve a closer approximation to the target final tooth position may include tooth translational and/or rotations movements that are considered over corrections, as they have a modified apparent final position that goes beyond the actual final position. In practice, the tooth movement and/or rotation for each tooth in the six degrees of freedom (e.g., buccal/lingual, mesial distal, and intrusion/extrusion) may be limited or constrained, to enhance safety. For example, FIGS. 10A and 10B illustrate a constrained (FIG. 10A) and unconstrained (FIG. 10B) example. In FIG. 10A the overtreatment movement/rotation is limited for each tooth to 1 mm/10 degrees. In FIG. 10B the overtreatment movement/rotation is unconstrained. Constraining the overtreatment (e.g., in the treatment plan modification engine, which may also be referred to herein as the overtreatment engine) may limit the aggressiveness of overtreatment and may prevent overly aggressive treatment movements.

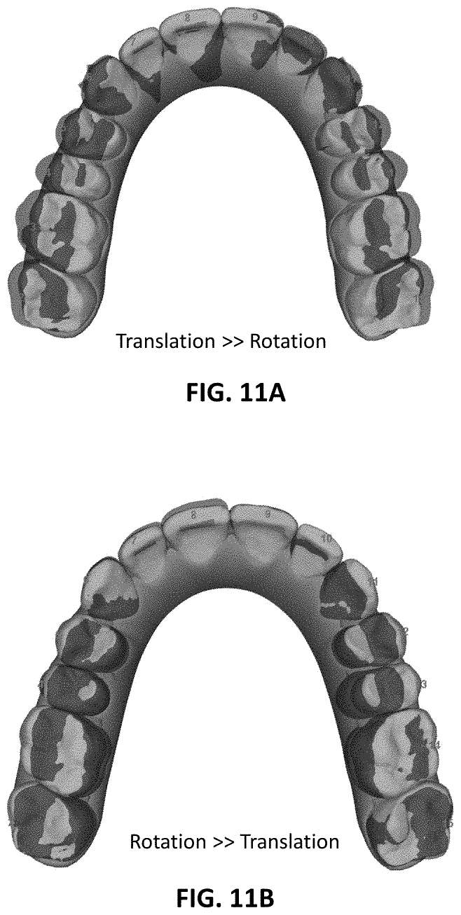

FIGS. 11A and 11B graphically illustrate weighting of the different movement/rotations of each tooth that may be accounted when estimating the difference indicator. For example, in FIG. 11A the translation of teeth is weighted much more than the rotation of teeth, while in FIG. 11B the rotation of the teeth is weighted much more than the translation.

The methods and apparatuses described herein illustrate general and accurate linear regression models (trained prediction models) to predict tooth movement. These methods and apparatuses have proven to be very effective, particularly when using information from combined movements & neighboring teeth. For example, the accuracy of predicting arch expansion (Tx) was approximately ˜80%, while the accuracy when predicting deep bite (Tz) was approximately ˜78%. Further, these techniques were both simple and interpretable. For example, tipping and reaction force effects emerged as model components. Any of these methods and apparatuses may be used to apply to optimize overtreatment in general, and may allow control of overtreatment and control of treatment goal weighting.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of generating or modifying a dental treatment plan for a patient having a set of initial patient tooth positions, a treatment plan and a set of target tooth positions, the method comprising:
selecting a trained prediction model, wherein the trained prediction model is trained, for each individual tooth of a dental arch, to include multiple translational and rotational directions for the individual tooth as well as reaction forces on the individual tooth due to one or more teeth that are adjacent to the individual tooth;
generating a set of predicted tooth positions from the set of initial patient tooth positions, the treatment plan, and the selected trained prediction model;
comparing the set of predicted tooth positions to the set of target tooth positions to determine a difference indicator;
if the difference indicator is at or greater than a threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and
outputting an optimized treatment plan based on the modified treatment plan.

2. The method of claim 1, further comprising generating a set of dental appliances based on the modified treatment plan.

3. The method of claim 1, wherein selecting the trained prediction model comprises selecting a trained neural network from a library of trained neural networks indexed by patient characteristics.

4. The method of claim 1, wherein selecting the trained prediction model comprises selecting the trained prediction model based on one or more patient characteristics including: the patient's age, the patient's gender, a therapeutic problem of the patient, and/or a cosmetic concern of the patient.

5. The method of claim 1, wherein selecting the trained prediction model comprises selecting a trained prediction model that is trained, for each individual tooth of a dental arch, using a linear regression model for each of the multiple translational and rotational directions.

6. The method of claim 5, wherein the trained prediction model is trained using a Huber linear regression model for each of the multiple translational and rotational directions.

7. The method of claim 1, wherein selecting the trained prediction model comprises selecting a trained prediction model that is trained for each individual tooth of the dental arch, to include multiple translational and rotational directions including six rotational and translational directions.

8. The method of claim 7, wherein the six rotational and translational directions include: buccal/lingual, mesial distal, and intrusion/extrusion.

9. The method of claim 1, wherein selecting the trained prediction model comprises selecting a trained prediction model that is trained for each individual tooth of the dental arch, to include multiple translational and rotational directions for the reaction forces.

10. The method of claim 9, wherein the multiple translational and rotational directions the reaction forces include six rotational and translational directions including: buccal/lingual, mesial distal, and intrusion/extrusion.

11. The method of claim 1, wherein comparing the set of predicted tooth positions to the set of target tooth positions comprises determining a difference for each of the multiple translational and rotational directions for each tooth and combining the differences for each of the multiple translational and rotational directions for each tooth to determine the difference indicator.

12. The method of claim 1, wherein comparing the set of predicted tooth positions to the set of target tooth positions comprises determining a difference for each of the multiple translational and rotational directions for each tooth, weighting all or some of the differences of the multiple translational and rotational directions for each tooth, and combining the weighted and any unweighted differences for each of the multiple translational and rotational directions for each tooth to determine the difference indicator.

13. The method of claim 12, further comprising receiving one or more weighting values specific to a clinician associated with the patient, wherein the one or more weighting values are used for weighting.

14. The method of claim 1, further comprising displaying, on a user display, an image of the target tooth positions and an image of the subject's teeth in the set of predicted tooth positions.

15. The method of claim 1, wherein the trained prediction model is trained, for each individual tooth of a dental arch, to include reaction forces on the individual tooth due to three or more teeth that are adjacent to the individual tooth.

16. A method of generating or modifying a dental treatment plan for a patient having a set of initial patient tooth positions, a treatment plan and a set of target tooth positions, the method comprising:
selecting a trained prediction model based on one or more patient characteristic, wherein the trained prediction model is trained, for each individual tooth of a dental arch, to include a linear regression for each of multiple translational and rotational directions for the individual tooth as well as for multiple translational and rotational directions of one or more teeth that are adjacent to the individual tooth;
generating a set of predicted tooth positions from the set of initial patient tooth positions, the treatment plan, and the selected trained prediction model;
comparing the set of predicted tooth positions to the set of target tooth positions for each of the multiple translational and rotational directions to determine a difference indicator;
if the difference indicator is less than a threshold value, outputting an optimized treatment plan based on the treatment plan;
if the difference indicator is at or greater than the threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and
outputting an optimized treatment plan based on the modified treatment plan.

17. A non-transitory computer-readable storage medium comprising instructions that, when executed by one or more processors of a device, cause the device to perform operations comprising:
selecting a trained prediction model based on one or more patient characteristic, wherein the trained prediction model is trained, for each individual tooth of a dental arch, to include multiple translational and rotational directions for the individual tooth as well as reaction forces on the individual tooth due to one or more teeth that are adjacent to the individual tooth;
generating a set of predicted tooth positions from a set of initial patient tooth positions, a treatment plan, and the selected trained prediction model;
comparing the set of predicted tooth positions to the set of target tooth positions to determine a difference indicator;
if the difference indicator is at or greater than a threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and
outputting an optimized treatment plan based on the modified treatment plan.

18. The non-transitory computer-readable storage medium of claim 17, wherein the instructions further cause the device to perform the operation of: generating a set of dental appliances based on the modified treatment plan.

19. The non-transitory computer-readable storage medium of claim 17, wherein selecting the trained prediction model comprises selecting a trained neural network from a library of trained neural networks indexed by patient characteristics.

20. The non-transitory computer-readable storage medium of claim 17, wherein selecting the trained prediction model comprises selecting the trained prediction model based on the one or more patient characteristics including: the patient's age, the patient's gender, a therapeutic problem of the patient, and/or a cosmetic concern of the patient.

21. The non-transitory computer-readable storage medium of claim 17, wherein selecting the trained prediction model comprises selecting a trained prediction model that is trained, for each individual tooth of a dental arch, using a linear regression model for each of the multiple translational and rotational directions.

22. The non-transitory computer-readable storage medium of claim 21, wherein the trained prediction model is trained using a Huber linear regression model for each of the multiple translational and rotational directions.

23. The non-transitory computer-readable storage medium of claim 16, wherein selecting the trained prediction model comprises selecting a trained prediction model that is trained for each individual tooth of the dental arch, to include multiple translational and rotational directions including six rotational and translational directions.

24. The non-transitory computer-readable storage medium of claim 23, wherein the six rotational and translational directions include: buccal/lingual, mesial distal, and intrusion/extrusion.

25. The non-transitory computer-readable storage medium of claim 17, wherein selecting the trained prediction model comprises selecting a trained prediction model that is trained for each individual tooth of the dental arch, to include multiple translational and rotational directions for the reaction forces.

26. The non-transitory computer-readable storage medium of claim 25, wherein the multiple translational and rotational directions the reaction forces include six rotational and translational directions including: buccal/lingual, mesial distal, and intrusion/extrusion.

27. The non-transitory computer-readable storage medium of claim 17, wherein comparing the set of predicted tooth positions to the set of target tooth positions comprises determining a difference for each of the multiple translational and rotational directions for each tooth and combining the differences for each of the multiple translational and rotational directions for each tooth to determine the difference indicator.

28. The non-transitory computer-readable storage medium of claim 17, wherein comparing the set of predicted tooth positions to the set of target tooth positions comprises determining a difference for each of the multiple translational and rotational directions for each tooth, weighting all or some of the multiple translational and rotational directions for each tooth, and combining the weighted and any unweighted differences for each of the multiple translational and rotational directions for each tooth to determine the difference indicator.

29. The non-transitory computer-readable storage medium of claim 28, wherein the instructions further cause the device to perform the operation of: receiving one or more weighting values specific to a clinician associated with the patient, wherein the one or more weighting values are used for weighting.

30. The non-transitory computer-readable storage medium of claim 17, wherein the instructions further cause the device to perform the operation of: displaying, on a user display, an image of the target tooth positions and an image of the subject's teeth in the set of predicted tooth positions.

31. A non-transitory computer-readable storage medium comprising instructions that, when executed by one or more processors of a device, cause the device to perform operations comprising:
selecting a trained prediction model based on one or more patient characteristic, wherein the trained prediction model is trained, for each individual tooth of a dental arch, to include a linear regression for each of multiple translational and rotational directions for the individual tooth as well as for multiple translational and rotational directions of one or more teeth that are adjacent to the individual tooth;
generating a set of predicted tooth positions from a set of initial patient tooth positions, a treatment plan, and the selected trained prediction model;
comparing the set of predicted tooth positions to the set of target tooth positions for each of the multiple translational and rotational directions to determine a difference indicator;
if the difference indicator is less than a threshold value, outputting an optimized treatment plan based on the treatment plan;
if the difference indicator is at or greater than the threshold value, iteratively modifying the treatment plan, and repeating the steps of generating the set of predicted tooth positions using the modified treatment plan as the treatment plan, and comparing the set of predicted tooth positions to the set of target tooth positions to determine the difference indicator, until the difference indicator is below the threshold value or a completion criterion is met; and
outputting an optimized treatment plan based on the modified treatment plan.
